(19) United States
(12) Patent Application Publication
(10) Pub. No.: US 2025/0144056 A1
(43) Pub. Date: May 8, 2025

(54) ANTIMICROBIAL HOST DEFENSE PEPTIDE-INDUCING COMPOSITIONS AND METHODS OF PRODUCTION AND USE THEREOF

(71) Applicant: Board of Regents for the Oklahoma Agricultural and Mechanical Colleges, Stillwater, OK (US)

(72) Inventors: Guolong ZHANG, Stillwater, OK (US); Melanie Anne WHITMORE, Stillwater, OK (US)

(21) Appl. No.: 18/834,102

(22) PCT Filed: Jan. 31, 2023
(86) PCT No.: PCT/US2023/061622

§ 371 Date: Jul. 29, 2024

Related U.S. Application Data
(60) Provisional application No. 63/304,997, filed on Jan. 31, 2022.

(51) Int. Cl. A61K 31/19; A61K 31/167; A61K 31/404; A61K 31/496; A61K 31/506; A61K 31/551; A61K 31/706; A61P 37/04

(52) U.S. Cl. CPC A61K 31/19; A61K 31/167; A61K 31/404; A61K 31/496; A61K 31/506; A61K 31/551; A61K 31/706; A61P 37/04

(57) ABSTRACT

Compositions are disclosed that include a combination of at least one histone deacetylase inhibitor with at least one DNA or histone methyltransferase inhibitor. The combination of the histone deacetylase inhibitor with the methyltransferase inhibitor synergistically induces antimicrobial host defense peptide gene expression.

### CROSS REFERENCE TO RELATED APPLICATIONS/INCORPORATION BY REFERENCE STATEMENT

The subject application claims benefit under 35 USC § 119 (e) of U.S. Provisional Application No. 63/304,997, filed Jan. 31, 2022. The entire contents of the above-referenced patent application(s) are hereby expressly incorporated herein by reference.

### STATEMENT REGARDING FEDERALLY SPONSORED RESEARCH OR DEVELOPMENT

This invention was made with U.S. Government support under USDA Grant Nos. 2018-68003-27462 and 2020-67016-31619 awarded by the Department of Agriculture. The United States Government has certain rights in this invention.

### BACKGROUND

Routine use of antibiotics at subtherapeutic levels in animal feed has been criticized as a major driving force for the emergence of antibiotic-resistant pathogens, a serious public health concern worldwide. Although various alternatives to antibiotics, including direct-fed microbials, botanical extracts, feed enzymes, bacteriophages, bacteriocins, and acidifiers have been explored in the livestock industry, none has been shown to match the cost-effectiveness, convenience, and performance offered by current conventional antibiotics. Unfortunately, most of the immunomodulators currently on the market nonspecifically stimulate a broad spectrum of immune and inflammatory responses, which often adversely affect animal growth and health.

Antimicrobial resistance is a global health crisis due to misuse and overuse of antimicrobials in both human and food animals (1, 2). Host defense peptides (HDPs) are an essential component of the innate immunity system by providing the first line of defense against bacterial, viral, fungal, and parasitic infections in almost all living organisms (3, 4). Two major families of HDPs, known as defensins and cathelicidins, exist in vertebrate animals (3, 4). There are a total of 14 β-defensins known as AvBD1-14 and four cathelicidins known as CATH1-3 and CATHB1 in chickens (5, 6). With a net positive charge and amphipathic structure, these small peptides kill pathogens primarily through electrostatic interactions and subsequent disruption and rupture of membranes (3, 4). HDPs have also been found to actively participate in chemotaxis, endotoxin neutralization, wound healing, and maintenance of barrier integrity (7, 8). Hence, utilization of HDPs against infections has emerged as a novel host-directed therapeutic approach (3, 4, 8, 9). Specifically, modulation of HDP synthesis is being explored as an antibiotic-alternative strategy for disease control and prevention (9-11).

Epigenetic modifications of histones or DNA have been found to profoundly impact gene transcription (12, 13). Histones are covalently modified in a variety of ways such as methylation and acetylation, while DNA is modified by methylation and demethylation (12, 13). Acetylation of histones occurs through histone acetyltransferases (HAT) and deacetylation through histone deacetylases (HDAC). Histones are also methylated by a group of histone methyltransferases (HMT) and demethylated by histone demethylases, while DNA is methylated by DNA methyltransferases (DNMT) and demethylated actively by various enzymes or passively during DNA replication (14). Hyperacetylation of histones as well as hypomethylation of DNA or histones is generally associated with active gene transcription, while histone hypoacetylation or DNA/histone hypermethylation often leads to transcription suppression (12, 13). HDAC inhibitors (HDACi) are classified into hydroximates, cyclic peptides, aliphatic acids, and benzamides based on their structural differences (15). A number of HDACi such as (but not limited to) butyrate are well known to induce HDP gene expression (10, 16), demonstrating a positive role of histone acetylation in HDP regulation. Several HDP genes have been found to be upregulated by DNA methyltransferase inhibitors (DNMTi) in human and bovine cells (17-19), and knockdown of a histone demethylase reduces HDP gene expression in human skin keratinocytes (20), implying the benefit of demethylating DNA and histones in HDP expression.

There is a need in the art for new and improved compositions that overcome the disadvantages and defects of the prior art. In particular, there is a need for new and improved compositions that confer on the host the ability to reduce the occurrence of infections without triggering inflammation and without inducing antimicrobial resistance, while also maintaining optimal animal health and performance. It is to such new and improved compositions, as well as methods of production and use thereof, that the present disclosure is directed.

### BRIEF DESCRIPTION OF THE DRAWINGS

FIG. 1 illustrates the structures of certain histone methyltransferase inhibitors (HMTi), DNA methyltransferase inhibitors (DNMTi), and histone deacetylase inhibitors (HDACi) used in accordance with the present disclosure. BIX01294 (hydrochloride hydrate) and A-366 are G9a-specific HMTi, while UNC1999 is a specific EZH1/2 HMTi.

FIG. 2 illustrates concentration- and time-dependent induction of AvBD9 mRNA expression by HMTi or DNMTi. (A) Fold changes in relative luciferase activity in stable chicken HTC/AvBD9-luc luciferase reporter cells in response to indicated concentrations (μM) of each compound for 24 h. (B) Fold changes in the AvBD9 mRNA expression level in chicken HTC cells stimulated with indicated concentrations of each compound for 24 h. (C) Fold changes in the AvBD9 mRNA expression level in chicken HTC cells stimulated with 10 UM of each compound for indicated lengths of time. AvBD9 mRNA expression was analyzed using RT-qPCR. Results are shown as means±SEM of three independent experiments. The bars not sharing a common superscript are considered significantly different (P<0.05) based on one-way analysis of variance and post hoc Tukey's test.

FIG. 3 illustrates the synergistic induction of luciferase activity in HTC/AvBD9-luc cells between butyrate and an HMTi or a DNMTi. Chicken HTC/AvBD9-luc luciferase reporter cells were stimulated in duplicate with indicated concentrations (μM) of BIX01294 (A), UNC1999 (B), or SGI-1027 (C), individually or in combination with 2 mM butyrate for 24 h, followed by luciferase assay for the AvBD9 gene promoter activity. Results are shown as means±SEM of three independent experiments. The bars not sharing a common superscript are considered significantly different (P<0.05) based on one-way analysis of variance and post hoc Tukey's test.

FIG. 4 illustrates the synergistic induction of the AvBD9 mRNA expression in HTC cells between HMTI and butyrate or between DNMTi and butyrate. Chicken HTC cells were stimulated in duplicate with indicated concentrations (μM) of BIX01294 (A), UNC1999 (B), SGI-1027 (C), or 5-azacytidine (D) individually or in combination with 2 mM butyrate for 24 h, followed by RT-qPCR analysis of AvBD9 mRNA expression. Results are shown as means±SEM of three independent experiments. The bars not sharing a common superscript are considered significantly different (P<0.05) based on one-way analysis of variance and post hoc Tukey's test.

FIG. 5 illustrates the synergistic induction of multiple HDP mRNA expression in HTC cells between BIX01294 and butyrate. Chicken HTC cells were stimulated in duplicate with indicated concentrations (M) of BIX01294 with or without 2 mM butyrate for 24 h, followed by RT-qPCR analysis of different chicken HDP genes. Results are shown as means±SEM of two independent experiments. The treatments not sharing a common superscript are considered significantly different (P<0.05) based on one-way analysis of variance and post hoc Tukey's test.

FIG. 6 illustrates the synergistic induction of the HDP mRNA expression in HD11 cells and peripheral blood mononuclear cells (PBMCs) between butyrate and an HMTI. Chicken HD11 macrophages were stimulated in duplicate with 2 mM butyrate with or without either 5 μM BIX01294 or 5 μM UNC1999 for 24 h, followed by RT-qPCR analysis of the gene expression levels of AvBD9 (A), AvBD1 (B), AvBD4 (C), AvBD10 (D), and CATH2 (E). (F) Chicken PBMCs were stimulated in duplicate with indicated concentrations (μM) of BIX01294 in the presence or absence of 1 mM butyrate for 24 h, followed by RT-qPCR of AvBD9 expression. Results are shown as means±SEM of 2-3 independent experiments. The treatments not sharing a common superscript are considered significantly different (P<0.05) based on one-way analysis of variance and post hoc Tukey's test.

FIG. 7 illustrates the synergistic induction of chicken HDP mRNA expression in HTC cells between HMTi and HDACi or between DNMTi and HDACi. Chicken HTC cells were stimulated in duplicate with an HMTi (2.5 or 5 μM BIX01294) in the presence or absence of one of three HDACi (5 M SAHA, 2 mM butyrate, or 2.5 μM mocetinostat) for 24 h, followed by RT-qPCR analysis of the expressions of AvBD9 (A) and other chicken HDP genes (B). (C) Chicken HTC cells were stimulated in duplicate with an HMTi (2 or 5 μM UNC1999 or 5 μM A-366) individually or in combination with an HDACi (2 mM butyrate or 2.5 μM mocetinostat) for 24 h, followed by RT-qPCR analysis of the expressions of AvBD9 gene. (D) Chicken HTC cells were stimulated in duplicate with a DNMTI (2.5 or 5 μM SGI-1027 or 5-azacytidine) individually or in combination with a HDACi (2 mM butyrate or 2.5 μM mocetinostat) for 24 h, followed by RT-qPCR analysis of AvBD9 expression. Results are shown as means±SEM of three independent experiments. The bars not sharing a common superscript are considered significantly different (P<0.05) based on one-way analysis of variance and post hoc Tukey's test.

FIG. 8 illustrates regulation of the genes involved in barrier function, host defense, and inflammation in HTC cells by HMTi and HDACi. Chicken HTC cells were stimulated in duplicate with 5 μM BIX01294 and 2 mM butyrate individually or in combination for 24 h, followed by 3-h stimulation with 10 ng/ml lipopolysaccharide (LPS) for another 3 h. RT-qPCR was performed to analyzed the expressions of CLND1 (A), MUC2 (B), AvBD9 (C), IL-16 (D), and IL-8 (E). (F) Chicken HTC cells were stimulated in duplicate with 5 μM UNC1999 or in combination with 2 mM butyrate or 2.5 μM mocetinostat for 24 h, followed by RT-qPCR analysis of CLND1 expression. Results are shown as means±SEM of three independent experiments. The bars not sharing a common superscript are considered significantly different (P<0.05) based on one-way analysis of variance and post hoc Tukey's test.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “polypeptide” as used herein will be understood to refer to a polymer of amino acids. The polymer may include d-, I-, or artificial variants of amino acids. In addition, the term “polypeptide” will be understood to include peptides, proteins, and glycoproteins.

The term “polynucleotide” as used herein will be understood to refer to a polymer of two or more nucleotides. Nucleotides, as used herein, will be understood to include deoxyribose nucleotides and/or ribose nucleotides, as well as artificial variants thereof. The term polynucleotide also includes single-stranded and double-stranded molecules.

The term “synergy” or “synergistic” means that the interaction of two or more substances to produce a combined effect is greater than the sum of their separate effects.

As used herein, a “structural” or “chemical” analog is a compound having a chemical structure similar to that of another one, but differing from it in respect of certain components, e.g., differing by one or more atoms, functional groups, or substructures, which are replaced with other atoms, groups, or substructures.

As used herein, a “functional” analog is a chemical compound that has properties or activities similar to those of another compound (e.g., physical, chemical, biochemical, or pharmacological properties such as mechanism or target of action), without necessarily sharing structural similarity, i.e., the chemical structures differ.

The terms “analog” or “variant” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all. Alternatively, and/or in addition thereto, for a chemical, an analog may be any structure that has the desired functionalities (including alterations or substitutions in the core moiety), even if comprised of different atoms or isomeric arrangements.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of infection by or growth of microbes and/or opportunistic infections. The effective amount for a subject will depend upon the type of subject, the subject's size and health, the method of administration, the duration of treatment, the specific formulations employed, the nature and severity of any condition/disease/infection present, the nature of concurrent therapy (if any), and the like. Thus, it is not possible to specify an exact effective amount in advance. However, the effective amount for a given situation can be determined by one of ordinary skill in the art using routine experimentation based on the information provided herein.

Turning now to the inventive concept(s), disclosed herein are compositions and methods, and in particular (but not by way of limitation), compositions and methods that induce expression of antimicrobial host defense peptide(s) in a subject. A synergistic effect on host defense peptide gene expression was observed when two to three compounds within or between different classes are combined in the compositions of the present disclosure.

The compositions of the present disclosure may be formulated for administration to a subject (such as, but not limited to, a human and/or an animal) via any formulation known in the art or otherwise contemplated herein that may be acceptable to the subject for consumption. For example (but not by way of limitation), the compositions may be a nutritional composition, such as, but not limited to, an animal feed, or a nutritional supplement (which can be administered by itself or added to food and/or water).

Certain non-limiting embodiments of the present disclosure are directed to a composition that comprises: (a) at least one histone deacetylase inhibitor; and (b) at least one methyltransferase inhibitor selected from a DNA methyltransferase inhibitor and/or a histone methyltransferase inhibitor.

Any histone deacetylase inhibitor (HDACi) known in the art or otherwise contemplated herein may be utilized as element (a) of the compositions of the present disclosure. Non-limiting examples of histone deacetylase inhibitors that may be utilized in accordance with the present disclosure include butyrate, phenylbutyrate, 4-phenylbutyric acid, β-hydroxybutyrate, vorinostat (suberoylanilide hydroxamic acid, SAHA), entinostat (MS-275), panobinostat (LBH589), mocetinostat (MGCD0103), belinostat (PXD101), romidepsin (FK228, depsipeptide), MC1568, tubastatin A, givinostat (ITF2357), dacinostat (LAQ824), CUDC-101, quisinostat (JNJ-26481585), pracinostat (SB939), PCI-34051, diallyl disulfide, depudecin, droxinostat, abexinostat (PCI-24781), RGFP966, AR-42, ricolinostat (ACY-1215), psammaplin A, tacedinaline (CI994), fimepinostat (CUDC-907), M344, tubacin, RG2833 (RGFP109), resminostat, BRD3308, SIS17, SR-4370, pimelic diphenylamide 106, NKL 22, azumamide E, tinostamustine (EDO-S101), SKLB-23bb, TH34, suberohydroxamic acid, UF010, WT161, valproic acid, 2-hexyl-4-pentynoic acid, tucidinostat (Chidamide), TMP195, TMP269, BRD73954, BG45, domatinostat (4SC-202), CAY10603, CAY10398, LMK-235, splitomicin, santacruzamate A (CAY10683), nexturastat A, HPOB, curcumin, divalproex sodium, scriptaid, GSK3117391, BML-210 (CAY10433), sulforaphane, raddeanin A, isoguanosine, sinapinic acid, tasquinimod, parthenolide, m-carboxycinnamic acid bis-hydroxamide, MC1742, JNJ-26481585, apicidin, HC toxin, oxamfaltin, bufexamac, KD5170, dihydrochlamydocin, 1-alaninechlamydocin, trapoxin A, microsporin A, largazole, sirtinol, inauhzin, SRT1720, SRT2104, SRT3025, CAY10591, CAY10602, thiomyristoyl, AGK2, JGB1741, salermide, AK-7, JFD00244, BML-278, pinosylvin, theobromine, EX-527, piceatannol, fisetin, vitexin, orientin, hyperforin, butein, ursolic acid, epicocconigrone, kaempferol, marein, zerumbone, lycorine, (S)-allylmercaptocysteine, and the like, as well as any combinations thereof.

The HDACi(s) may be present in the composition at any percentage of concentration that allows the HDACi(s) to function as described or as otherwise contemplated herein. For example (but not by way of limitation), the HDACi(s) may be present in a sufficient amount to induce expression of one or more host defense peptides (either alone or in combination with one or more methyltransferases). In certain particular (but non-limiting) embodiments, the HDACi(s) is present in the composition at a percent concentration of about 0.001%, about 0.005%, about 0.01%, about 0.05%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In addition, the scope of the presently disclosure also includes the presence of the HDACi(s) in the composition at any percent concentration that falls within any range formed from the combination of two values listed above (for example, a range of from about 1% to about 99%, a range of from about 2% to about 80%, a range of from about 3% to about 60%, a range of from about 10% to about 95%, a range of from about 40% to about 75%, etc.).

Any DNA methyltransferase inhibitor(s) known in the art or otherwise contemplated herein may be utilized alone or in combination with one or more compounds as element (b) of the compositions of the present disclosure. Non-limiting examples of DNA methyltransferase inhibitors that may be utilized in accordance with the present disclosure include decitabine, azacitidine, 2′-deoxy-5-fluorocytidine, thioguanine, zebularine, gamma-oryzanol, β-thujaplicin, procainamide, bobcat339, SGI-1027, RG108, RSC-133, SW155246, genistein, ellagic acid, rosmarinic acid, and the like, as well as any combinations thereof.

When present, the HMTi(s) may be present in the composition at any percentage of concentration that allows the HMTi(s) to function as described or as otherwise contemplated herein. For example (but not by way of limitation), the HMTi(s) may be present in a sufficient amount to induce expression of one or more host defense peptides (either alone or in combination with the HDACi(s)). In certain particular (but non-limiting) embodiments, the HMTi(s) is present in the composition at a percent concentration of about 0.001%, about 0.005%, about 0.01%, about 0.05%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In addition, the scope of the presently disclosure also includes the presence of the HMTi(s) in the composition at any percent concentration that falls within any range formed from the combination of two values listed above (for example, a range of from about 1% to about 99%, a range of from about 2% to about 80%, a range of from about 3% to about 60%, a range of from about 10% to about 95%, a range of from about 40% to about 75%, etc.).

Any histone methyltransferase inhibitor(s) known in the art or otherwise contemplated herein may be utilized alone or in combination with one or more compounds as element (b) of the compositions of the present disclosure. Non-limiting examples of histone methyltransferase inhibitors that may be utilized in accordance with the present disclosure include BIX01294, UNC0224, UNC0321, UNC0631, UNC0638, UNC0642, UNC0646, UNC1999, UNC6852, BRD9539, BRD4770, sinefungin, tazemetostat, nanaomycin A, lirametostat, 3-deazaneplanocin A, chaetocin, A-366, EPZ005687, EPZ011989, EPZ6438, GSK126, GSK343, GSK503, CM-272, CPI-169, CPI-360, EBI-2511, MS1943, JQEZ05, El1, (R)-PFI-2, PF-06726304, ZLD1039, and the like, as well as any combinations thereof.

When present, the DNMTi(s) may be present in the composition at any percentage of concentration that allows the DNMTi(s) to function as described or as otherwise contemplated herein. For example (but not by way of limitation), the DNMTi(s) may be present in a sufficient amount to induce expression of one or more host defense peptides (either alone or in combination with the HDACi(s)). In certain particular (but non-limiting) embodiments, the DNMTi(s) is present in the composition at a percent concentration of about 0.001%, about 0.005%, about 0.01%, about 0.05%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In addition, the scope of the presently disclosure also includes the presence of the DNMTi(s) in the composition at any percent concentration that falls within any range formed from the combination of two values listed above (for example, a range of from about 1% to about 99%, a range of from about 2% to about 80%, a range of from about 3% to about 60%, a range of from about 10% to about 95%, a range of from about 40% to about 75%, etc.).

Accordingly, in some aspects, the compositions described herein comprise at least two of the compounds described herein, i.e., two or more different compounds (such as 2, 3, 4, 5, 6, 7, 8, 9, 10 or more) in a single formulation. Accordingly, the present disclosure encompasses such formulations/compositions. The two or more compounds are generally substantially purified and are generally present in a pharmaceutically acceptable carrier or excipient, which may be aqueous or oil-based. In some aspects, such compositions are prepared as liquid solutions or suspensions, or as solid forms such as tablets, pills, powders, and the like. Solid forms suitable for solution in, or suspension in, liquids prior to administration are also contemplated (e.g., dried, desiccated, or lyophilized forms of the combinations), as are emulsified preparations. In some aspects, the liquid formulations are aqueous or oil-based suspensions or solutions. In some aspects, the active ingredients are mixed with excipients which are pharmaceutically acceptable and compatible with the active ingredients, e.g., pharmaceutically acceptable salts. Suitable excipients or carriers include, for example, water, ethanol, saline, DMSO, dextrose, glycerol, starch, limestone, microspheres, nanoparticles and the like, or combinations thereof. In addition, the composition may contain minor amounts of auxiliary substances such as wetting or emulsifying agents (such as vegetable oils, liposomes, and polymeric micelles), pH buffering agents, preservatives, and the like. In some aspects, the compounds are crude extracts of certain plants or animals.

Generally, when the subject is a non-human animal, the compositions are mixed with or added to animal feed, either during commercial preparation of the feed, or added to the feed as a supplement prior to providing the feed to the animal. The feed may be solid or liquid, e.g., adult subjects generally eat solid feed but young subjects (e.g., newborns) may need liquid food. Alternatively, the compositions may be added to drinking water, or administered by any other suitable route, e.g., by continual drinking or individual dosing as described above.

If it is desired to administer an oral form of the composition, or to provide a form that is suitable for mixing with animal feed, various thickeners, flavorings, diluents, emulsifiers, dispersing aids, bulking agents, or binders and the like, or combinations thereof, may be added. The composition may contain any such additional ingredients so as to provide the composition in a form suitable for administration, either directly or via a food product. The final amount of compound in the formulations varies, but is generally from about 1-99%. Still other suitable formulations for use in the present disclosure are found, for example in Remington's Science and Practice of Pharmacy, 22nd ed. (Pharmaceutical Press 2012, ISBN: 0857110624).

Some examples of materials which can serve as pharmaceutically acceptable carriers or excipients include, but are not limited to, ion exchangers, alumina, aluminum stearate, lecithin, serum proteins (such as human serum albumin), buffer substances (such as Tween® 80, phosphates, glycine, sorbic acid, or potassium sorbate), vegetable oils, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” refers to the relatively non-toxic, inorganic, and organic acid addition salts, and base addition salts, of compounds of the present disclosure. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al. (“Pharmaceutical Salts,” J. Pharm. Sci. (1977) 66:1-19), the entirety of which is expressly incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred (but not by way of limitation). Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

Certain particular (but non-limiting) embodiments of the compositions of the present disclosure include the following: a composition where (a) is butyrate, and (b) is selected from the group consisting of BIX01294, A-366, and UNC1999; a composition where (a) is butyrate, and (b) is selected from the group consisting of SGI-1027 and AZA; a composition where (a) is vorinostat, and (b) is BIX01294; a composition where (a) is mocetinostat, and (b) is of BIX01294, A-366, and UNC1999; and a composition where (a) is mocetinostat, and (b) is selected from the group consisting of SGI-1027 and AZA. However, it will be understood that the above compositions are provided for the purposes of illustration only; the compositions of the present disclosure may include any of the element(s) of (a) and any of the element(s) of (b) disclosed or otherwise contemplated herein.

Certain non-limiting embodiments of the present disclosure are directed to a method of inducing expression of at least one antimicrobial host defense peptide. In the method, at least one cell is contacted with any of the compositions disclosed herein above or otherwise contemplated herein under conditions that allow for expression of the at least one antimicrobial host defense peptide.

The compositions may induce expression of any antimicrobial host defense peptides known in the art or otherwise contemplated herein. Non-limiting examples of antimicrobial host defense peptides that can be induced in accordance with the present disclosure include avian β-defensins (AvBD) and cathelicidins (CATH), such as, but not limited to: AvBD1, AvBD2, AvBD3, AvBD4, AvBD5, AvBD6, AvBD7, AvBD8, AvBD9, AvBD10, AvBD11, AvBD12, AvBD13, AvBD14, CATH1, CATH2, CATH3, CATHB1, and the like, as well as any combinations thereof.

Certain non-limiting embodiments of the present disclosure are directed to a method of administering any of the compositions disclosed herein above or otherwise contemplated herein to an animal.

The agents described herein exhibit synergy with respect to increasing the level of expression of one or more genes encoding an antimicrobial host defense peptide, when administered in the combinations described herein. Generally, the combinations are administered as a single composition, i.e., the composition is a mixture of the two or more agents. However, administration of the agents separately is also encompassed, in which case administration is generally coordinated so that the subject takes in all the agents in the combination at least during a single day or one week period, and usually within a shorter time frame, e.g., within at least 10, 9, 8, 7, 6, 5, 4, 3, 2, or 1 hour(s) or less, so the agents are present together within the subject at the same time, the physiological activity of each is carried out at the same or at least at overlapping times, and the synergistic effect is thus realized. For example, one agent may be present in dry feed and the second agent may be present in drinking water, etc., both of which are consumed throughout the day. Or, if the subject is a human or a non-human animal that can be dosed by hand, the components of a combination may be administered one after the other, e.g., in two separate preparations. For example, one component may require an aqueous carrier and a second and/or third component may require an oil-based carrier, so that they cannot, or cannot easily, be combined into a single preparation. However, it is generally preferred (but not by way of limitation) to administer all agents of a combination at once as a single composition.

As such, certain non-limiting embodiments of the present disclosure also include a method of administering to a subject, either simultaneously or wholly or partially sequentially: (a) at least one of any of the histone deacetylase inhibitors disclosed or otherwise contemplated herein; and (b) at least one of any of the methyltransferase inhibitors selected from a DNA methyltransferase inhibitor and/or a histone methyltransferase inhibitor disclosed or otherwise contemplated herein.

The various methods of the present disclosure further include the step of optionally repeating any of the administration steps one or more times, as needed, and as described in detail herein above or otherwise contemplated herein.

The compositions may be administered in vivo by any suitable route including but not limited to: by inclusion in a food product, by inoculation or injection (e.g., intravenous, intraperitoneal, intramuscular, subcutaneous, and the like), by topical application and by absorption through epithelial or mucocutaneous linings. Other suitable means include but are not limited to: inhalation (e.g., as a mist or spray), orally (e.g., as a pill, capsule, liquid, etc.), intravaginally, intranasally, rectally, by ingestion of a food or probiotic product containing the combinations of agents, as eye drops, incorporated into dressings or bandages (e.g., lyophilized forms may be included directly in the dressing), etc. In some aspects, for non-human animals, administration is via a food product or drinking water. For a human subject, the mode of administration is typically oral or by injection. In addition, the compositions may be administered in conjunction with other treatment modalities such as substances that boost the immune system, various chemotherapeutic agents, one or more antibiotic agents (e.g., conventional antibiotics, but generally at a lower dose than when administered without the combinations described herein), vitamins, minerals, and the like.

Suitable subjects which would benefit by administration of the compositions described herein include but are not limited to various vertebrate animals such as fishes, amphibians, reptiles, birds and mammals, including both human and non-human mammals. The fishes, birds, and non-human mammals include those which are raised for commercial purposes, either to serve as food (e.g., meat) or to produce a product that serves as food (milk, eggs, etc.) or some other purpose, e.g., animals that are used for breeding, as companions or hobby pets, as show animals, for work or entertainment (e.g., sports), etc. The non-human subject may be domestic or wild, and/or may be located in a protected area such as a sanctuary or a zoo. Examples of suitable fish species that may be treated as described herein include but are not limited to: shellfish (including clams, crab, lobster, oysters, scallops and shrimp), trout and other stocking and food fish such as catfish, trout, salmon, walleyes, tilapia, bass, carp, bluegills, sunfish, perch, and eel. Examples of suitable avian species that may be treated as described herein include but are not limited to: chickens, ducks, geese, turkeys, guinea fowl, ostriches, pigeons, quails, and pheasants. Examples of suitable non-human mammals include but are not limited to: pigs, cattle, horses, sheep, goats, mice, rats, rabbits, guinea pigs, llamas, alpaca, addax, bison, camel, deer, donkey, eland, elk, gayal, mule, moose, oryx, water buffalo, yak, and zebu. In some aspects, the subject is a human.

The amount or dose of the combinations that is administered varies based on several factors, as will be understood by those of skill in the art. For example, the dose and frequency of administration varies according to the composition that is administered, the gender, age, weight, general physical condition, genetic background, etc. of the recipient. Generally, the dose will be in the range of from about 0.01 to about 1000 mg/kg of body weight or feed per day (e.g., about 0.1, 0.5, 1.0, 10, 20, 30, 40, 50, 60, 70, 80, 90, 100, 125, 150, 175, 200, 225, 250, 275, 300, 325, 350, 375, 400, 425, 450, 475 or 500 mg/kg, etc.).

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

### Example 1

Antimicrobial host defense peptides (HDPs) are an integral part of the innate immune system, acting as the first line of defense. Modulation of HDP synthesis has emerged as a promising host-directed approach to fight against infections. Inhibition of histone deacetylation or DNA methylation have been found to enhance HDP gene expression separately. In this Example, a possible synergy in HDP gene induction between histone deacetylase inhibitors (HDACi) and DNA/histone methyltransferase inhibitors (DNMTi/HMTi) was explored. Two chicken macrophage cell lines were treated with structurally distinct HDACi, HMTi, or DNMTi, individually or in various combinations, followed by HDP gene expression analysis. Each epigenetic compound was found to be capable of inducing HDP expression. Surprisingly, a combination of HDACi and HMTi or HDACi and DNMTi showed a strong synergy to induce the expressions of most HDP genes. The HDP-inducing synergy between butyrate, an HDACi, and BIX01294, an HMTi, was further verified in chicken peripheral blood mononuclear cells. Furthermore, tight junction proteins such as claudin 1 were also synergistically induced by HDACi and HMTi. Overall, it was concluded that HDP genes are regulated by epigenetic modifications. Strategies to increase histone acetylation while reducing DNA or histone methylation exert a synergistic effect on HDP induction and, therefore, allow for the control and prevention of infectious diseases.

### MATERIALS AND METHODS

Cell culture media and chemicals. RPMI 1640 medium, penicillin, streptomycin, and puromycin were purchased from Hyclone (Logan, UT), while fetal bovine serum (FBS) was obtained from Atlanta Biologicals (Lawrenceville, GA). Sodium butyrate and bacterial lipopolysaccharide (LPS) from Escherichia coli 055: B5 were procured from Millipore Sigma (St. Louis, MO), and BIX01294, A-366, UNC1999, SGI-1027, 5-azacytidine (AZA), vorinostat, and mocetinostat were all acquired from Cayman Chemical (Ann Arbor, Michigan).

Sodium butyrate and LPS were dissolved in RPMI 1640, while all other chemicals were dissolved in dimethyl sulfoxide (DMSO). In all subsequent cell culture experiments, cells treated with an equal volume of RPMI 1640 or DMSO were used as negative controls to the cells treated with individual compounds or their combinations.

Culture and stimulation of macrophages. Two different chicken macrophage cell lines, HTC (24) and HD11 (25), were kindly provided by Dr. Narayan C. Rath and Dr. Hyun Lillehoj of USDA-ARS and maintained in RPMI 1640 containing 10% heat-inactivated FBS, 100 U/ml penicillin, and 100 μg/ml streptomycin and subcultured every 3-4 days. After overnight seeding at 1.5×10⁶ cells/well in 6-well cell culture plates, cells were treated with different concentrations of each compound for 24 h or with the indicated concentration of each compound for various lengths of time for dose-response and time-course experiments, respectively. To study the synergy between HMTi/DNMTi and HDACi, different concentrations of an HMTi or DNMTi were added to the cells in the presence or absence of an HDACi for 24 h, followed by RNA isolation and HDP gene expression analysis as described below. HTC cells were also stimulated with butyrate, BIX01294, or their combination for 24 h, followed by 3-h stimulation with 10 ng/ml LPS and subsequent expression analysis.

Culture and stimulation of a stable HTC/AvBD9-luc luciferase reporter cell line. A stable HTC/AvBD9-luc cell line was generated by cloning a 2.0-Kb AvBD9 gene promoter fragment into a lentiviral luciferase reporter vector, pGreenFire1-mCMV-Puro (System Biosciences, Palo Alto, CA) and then transfected into HTC cells as previously described (22). The stable cell line was maintained in RPMI 1640 containing 10% heat-inactivated FBS, 100 U/ml penicillin, 100 μg/mL streptomycin, and 0.5 μg/mL puromycin and subcultured every 3-4 days. To measure transcriptional activation of the AvBD9 gene promoter in response to different epigenetic compounds, cells were seeded at 4×10⁴ cells/well in white 96-well tissue culture plates and stimulated in duplicate with different compounds individually or in combination for 24 h. An equal amount of a solvent was applied as a negative control. Luciferase activity was measured using STEADY-GLO® Luciferase Assay System (Promega, Madison, WI) on an L-Max II luminescence microplate reader (Molecular Devices, Sunnyvale, CA). Cell viabilities were assessed by adding alamarBlue Reagent (ThermoFisher, Waltham, MA) to cell culture 4 h before luciferase assay. Relative luciferase activity was determined for each well after normalization to the cell viability as previously described (22).

Isolation, culture, and stimulation of chicken peripheral blood mononuclear cells (PBMCs). Chicken PBMCs were isolated from EDTA-anticoagulated venous blood collected from 2- to 4-week-old broiler chickens through gradient centrifugation using Histopaque 1077 (Millipore Sigma) as previously described (22, 26). PBMCs were seeded at 3×10⁷ cells/well in 6-well cell culture plates and stimulated with different concentrations of an HMTi or DNMTi with or without 1 mM butyrate for 24 h, followed by RNA isolation and HDP gene expression analysis as described below.

Total RNA extraction and gene expression analysis. Total RNA was extracted using RNAzol (Molecular Biology Research Center, Cincinnati, OH) according to the manufacturer's protocol. Gene expression was performed using reverse transcription-quantitative PCR (RT-qPCR) as previously described (27-29). Reverse transcription was performed with 300 ng of total RNA and Maxima® First Strand cDNA Synthesis Kit (ThermoFisher Scientific, Pittsburgh, PA). The cDNA was then diluted 10-fold with RNase-free water prior to real-time PCR using QuantiTect SYBR® Green PCR Kit (Qiagen, Valencia, CA) in 10-μl reactions using primers specific for HDP, barrier function, and inflammatory cytokine genes as described (27, 29, 30). PCR was run in CFX Connect Real-Time PCR System (Bio-Rad, Hercules, CA) using the following program: 95° C. for 10 min, followed by 40 cycles of 94° C. for 15 sec, 55° C. for 20 sec, and 72° C. for 30 sec. The specificity of PCR reactions was confirmed by the melting curve analysis. The glyceraldehyde-3-phosphate dehydrogenase (GAPDH) gene was used as the reference for data normalization. The fold changes in gene expression in the cells treated with individual compounds or in combinations were calculated relative to the cells treated with an equal volume of RPMI 1640 or DMSO using the ΔΔCt method (31).

Statistical analysis. Data analysis and visualization were implemented in GraphPad Prism (GraphPad Software, La Jolla, CA). Results were presented as means±standard error of the mean (SEM) and compared among treatments using one-way analysis of variance and post hoc Tukey's test. Statistical significance was considered if P<0.05.

### Results

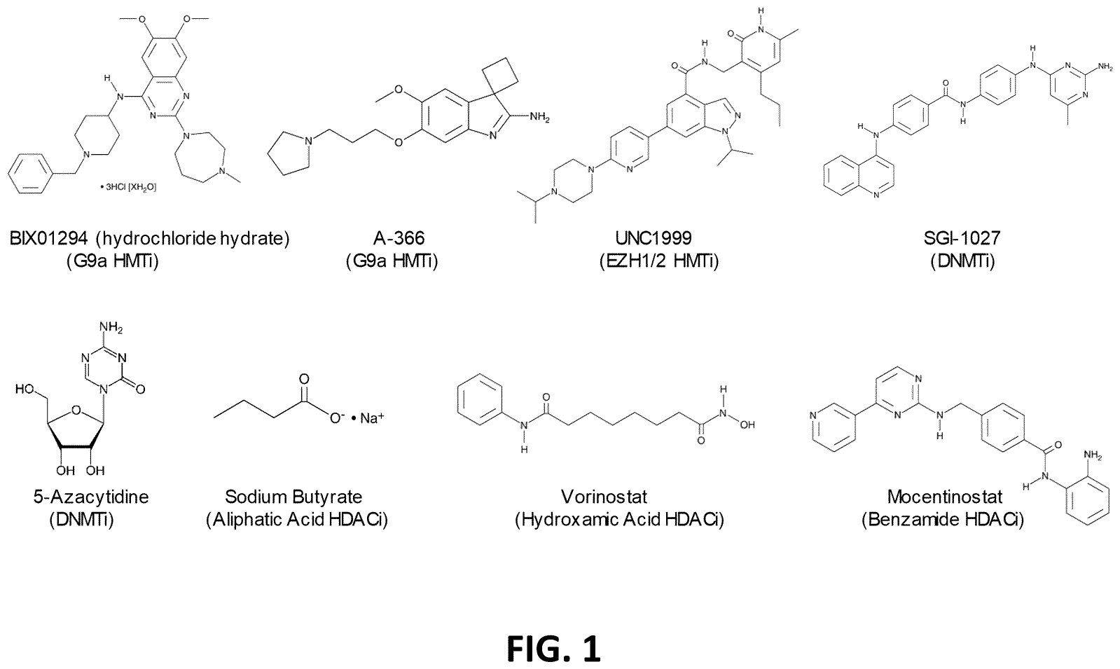

HMTi and DNMTi induce AvBD9 gene expression in concentration- and time-dependent manners. A number of epigenetic compounds including HDACi, HMTI, and DNMTI that are capable of inducing HDP gene expression have been identified using luciferase reporter cell lines driven by HDP gene promoters (21, 22). HDACi are well-known HDP inducers (10, 16). Three structurally distinct HMTi (BIX01294, A-366, and UNC1999), two DNMTi (SGI-1027 and AZA), and three HDACi (butyrate, virinostat, and mocentinostat) were selected to study their possible synergy in HDP gene induction. Their structures are shown in FIG. 1.

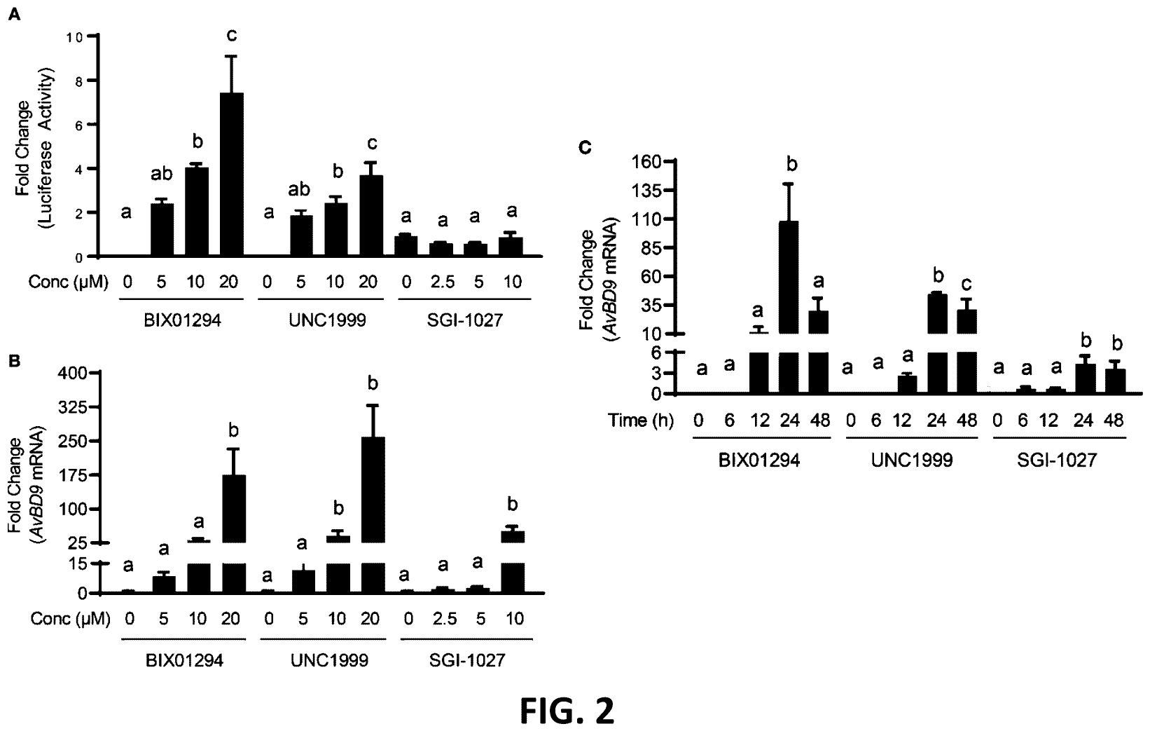

To further evaluate whether newly-identified HMTi (BIX01294 and UNC1999) and a DNMTi (SGI-1027) can induce HDP gene expression in both concentration- and dose-dependent manners, a chicken HTC/AvBD9-luc reporter cell line was initially treated with different concentrations of each compound for 24 h. BIX01294 and UNC1999 showed an obvious concentration-dependent activation of the AvBD9 gene promoter in the reporter cell line as indicated by increases in relative luciferase activity, but SGI-1027 was a weak activator (FIG. 2, Panel A). BIX01294 and UNC1999 were further confirmed to induce AvBD9 mRNA expression in the chicken HTC macrophage cell line in a concentration-dependent fashion, peaking with a 175- and 259-fold increase in response to 20 μM of each compound, respectively, while 10 μM SGI-1027 gave a 51-fold induction (FIG. 2, Panel B). Because it triggered a robust AvBD9 mRNA induction with each compound, 10 μM was used in subsequent time-course experiments. All three compounds also showed a time-dependent induction of AvBD9 mRNA expression in HTC cells, peaking at 24 h (FIG. 2, Panel C). Therefore, cells were treated for 24 h for all downstream analyses of HDP gene expression.

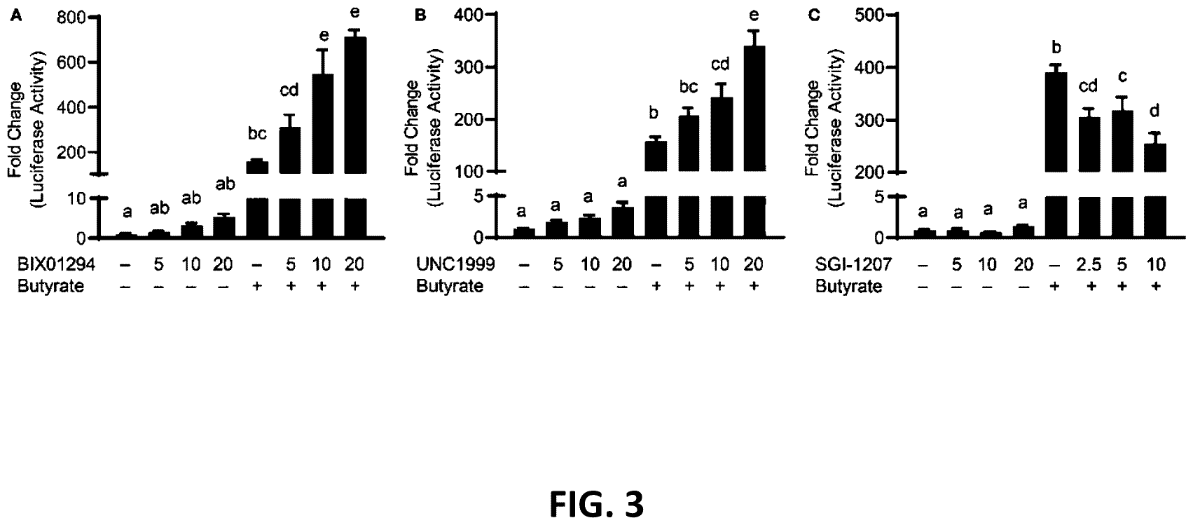

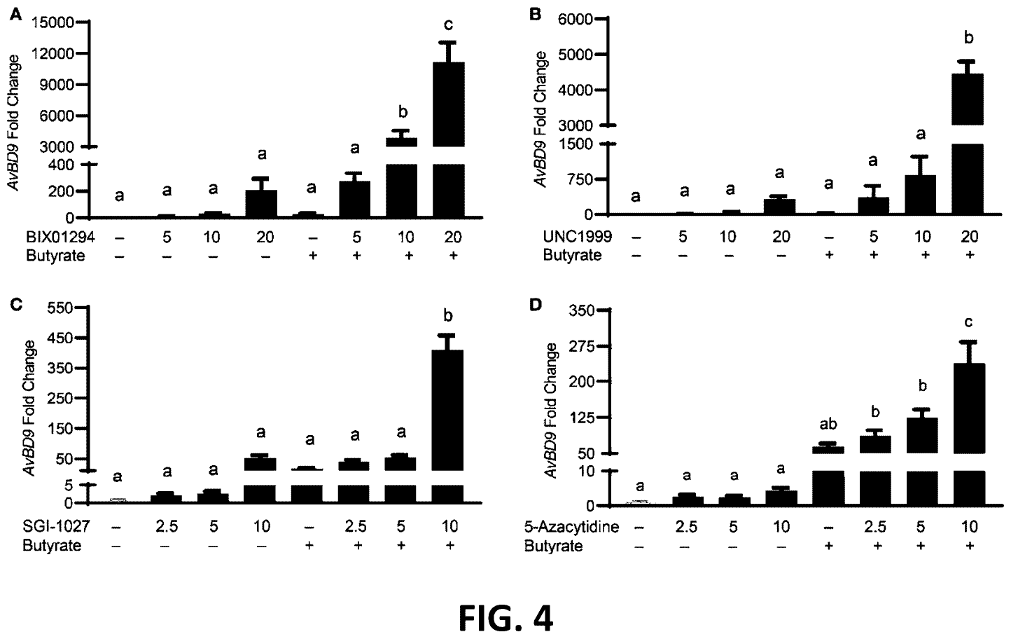

HMTi or DNMTi synergizes with butyrate to induce HDP gene expression. Butyrate, an HDACi, is well-known to induce HDP gene expression in different animal species including humans and chickens (26, 32). Sodium butyrate at 2 mM gives optimal HDP gene induction in chicken HTC and HD11 cells and further synergizes strongly with several other HDP-inducing compounds such as forskolin (27, 30), sugars (28), and cyclooxygenase-2 inhibitors (33). To explore a possible synergy between butyrate and an HMTi or DNMTi, HTC/AvBD9-luc reporter cells were treated with different concentrations of BIX01294, UNC1999, or SGI-1027 in the presence or absence of 2 mM butyrate for 24 h. A marked dose-dependent increase in the synergy in luciferase activity was observed between butyrate and BIX01294 (FIG. 3, Panel A) as well as between butyrate and UNC1999 (FIG. 3, Panel B). However, no synergy between butyrate and SGI-1027 was observed in the luciferase reporter cell line (FIG. 3, Panel C). HTC cells were used to confirm whether the synergy occurs between butyrate and an HMTi or DNMTi to induce AvBD9 mRNA expression. A concentration-dependent increase in AvBD9 mRNA expression was indeed observed between butyrate and BIX01294, UNC1999, or SGI-1027. For example, 20 μM BIX01294 and butyrate individually induced AvBD9 expression by 207- and 27-fold, respectively; however, a combination of 20 μM BIX01294 and butyrate gave a remarkable 11,165-fold increase in AvBD9 expression, which is an additional 54-fold increase over 20 μM BIX01294 alone (FIG. 4, Panel A). Similarly, 20 μM UNC1999 enhanced AvBD9 expression by 324-fold, but a UNC1999/butyrate combination gave a 4,470-fold increase, which reflected an additional 14-fold increase over UNC1999 alone (FIG. 4, Panel B). SGI-1027 at 10 UM augmented AvBD9 expression by 51-fold, but further induced AvBD9 by 411-fold when combined with butyrate, which is an additional 8-fold increase over 10 UM SGI-1027 alone (FIG. 4, Panel C). AZA, another well-known DNMTi (34), was also examined and confirmed its synergy with butyrate in a concentration-dependent manner (FIG. 4, Panel D). It is noted that the AvBD9-inducing synergy between butyrate and an HMTi tended to be stronger than between butyrate and a DNMTi.

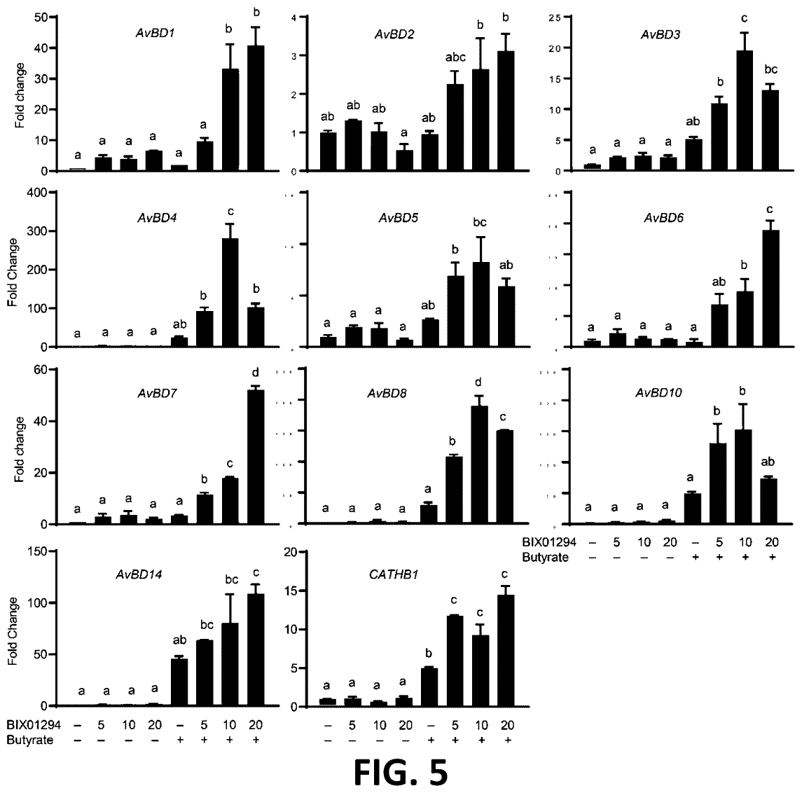

Moreover, among those HDP genes that are detectable in HTC cells, all were synergistically induced in response to both BIX01294 and butyrate, although different magnitudes of induction were observed with different HDP genes. For example, while AvBD2 was only upregulated approximately 2- to 3-fold by a combination of butyrate and BIX01294, AvBD4, AvBD8, AvBD10, and AvBD14 were induced by greater than 100-fold in response to butyrate and at least one concentration of BIX01294, with the remaining HDP genes showing an intermediate response (FIG. 5).

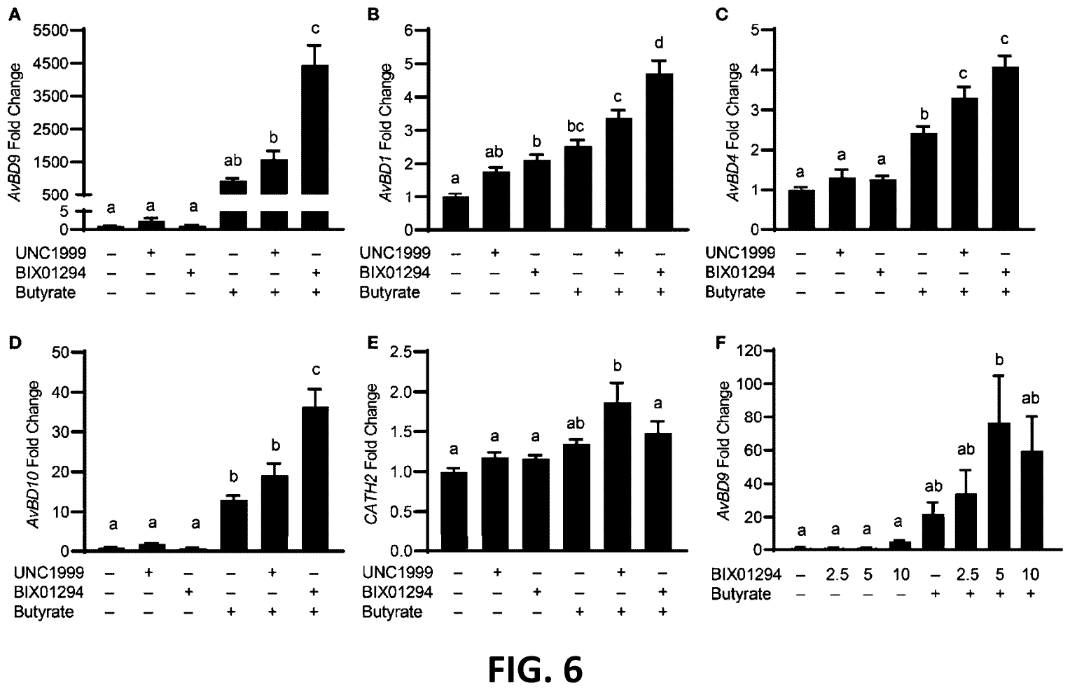

To confirm whether the HDP-inducing synergy between BIX01294 and butyrate is preserved in a different cell line, chicken HD11 macrophages were stimulated with butyrate in the presence or absence of BIX01294 or UNC1999 for 24 h. Consistent with the results in HTC cells, a drastic synergy in AvBD9 induction was observed between butyrate and BIX01294 and between butyrate and UNC1999 (FIG. 6, Panel A). Similarly, AvBD1 (FIG. 6, Panel B), AvBD4 (FIG. 6, Panel C), and AvBD10 (FIG. 6, Panel D) were also synergistically induced, albeit at lower magnitudes. On the other hand, CATH2 mRNA was minimally regulated by individual compounds or their combinations in HD11 cells (FIG. 6, Panel E). To further verify a possible HDP-inducing synergy between butyrate and BIX01294 in primary cells, chicken PBMCs were isolated and treated with BIX01294 in the presence or absence of butyrate for 24 h. BIX01294 alone induced AvBD9 in a dose-dependent manner, and the BIX01294/butyrate combination showed an obvious synergy (FIG. 6, Panel F).

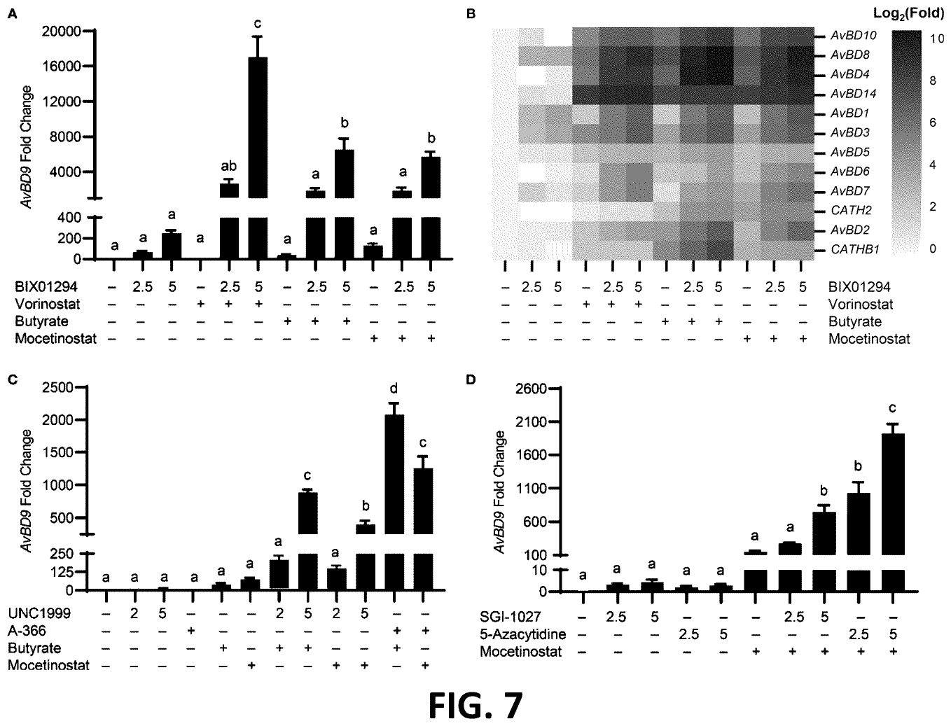

HMTi or DNMTi synergizes with HDACi to induce HDP gene expression. To further examine whether other structurally different HDACi also synergize with BIX01294 in AvBD9 induction, chicken HTC cells were treated with different concentrations of BIX01294 with or without an HDACi (vorinostat, butyrate, and mocetinostat; FIG. 1). A dramatic synergy in AvBD9 induction was observed between BIX01294 and any of three HDACi. For example, 5 μM BIX01294 in combination with vorinostat, butyrate, and mocetinostat gave an additional 69-, 26-, and 23-fold increase in AvBD9 gene expression over BIX01294 alone, respectively (FIG. 7, Panel A). In addition to AvBD9, most other HDP genes were also synergistically induced in response to a combination of BIX01294 and any of the three HDACi (FIG. 7, Panel B). To study whether other structurally distinct HMTi also synergize with HDACi in HDP induction, HTC cells were treated with UNC1999 or A-366 (FIG. 1) in the presence or absence of butyrate or mocetinostat. Indeed, a marked synergy was seen between any two-compound combination of an HMTi (UNC1999 or A-366) and an HDACi (butyrate or mocetinostat) (FIG. 7, Panel C).

Butyrate was shown to synergize in HDP gene induction with SGI-1027 and AZA (FIG. 4, Panels C and D). Thus, a possible synergy between a DNMTi and a structurally different HDACi was further examined. A drastic synergy was observed between mocetinostat and either DNMTi. For example, 5 μM SGI-1027, 5 μM AZA, and 2.5 μM mocetinostat induced AvBD9 gene expression by 4-, 3-, and 152-fold, respectively, while a combination of 5 μM SGI-1027/mocetinostat and 5 μM AZA/mocetinostat gave 747- and 1,925-fold increases in AvBD9 expression (FIG. 7, Panel D).

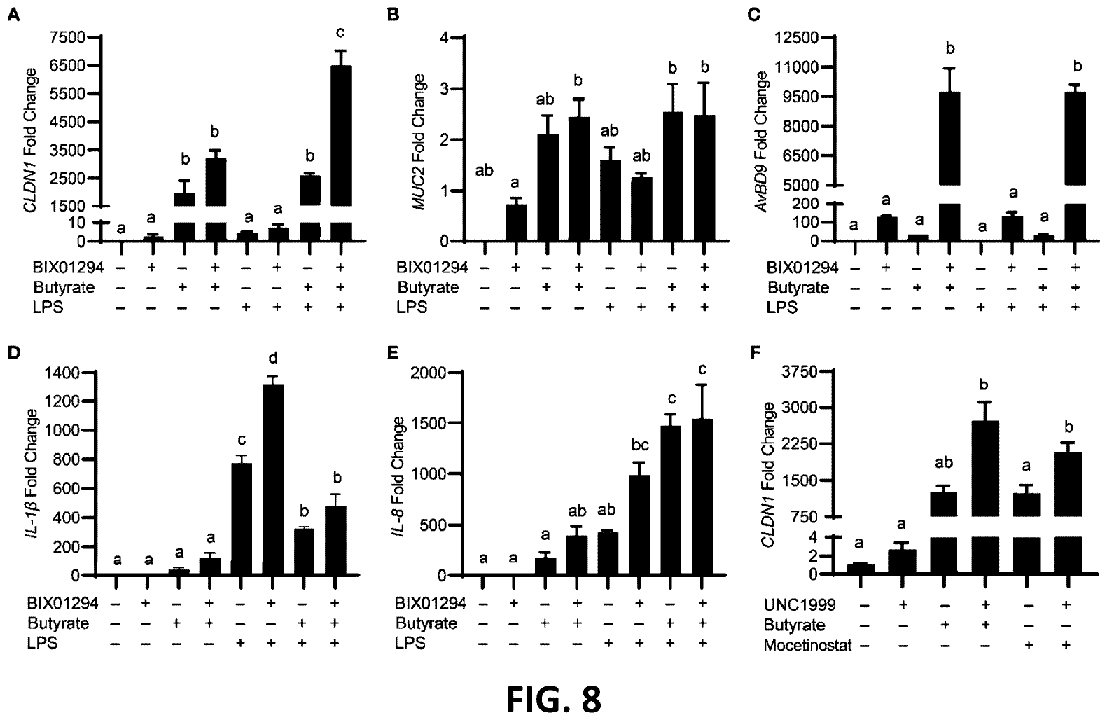

HMTi and butyrate cooperate to regulate the genes involved in barrier function and inflammation. Butyrate is known to enhance mucosal barrier function by inducing the genes for mucin 2 (MUC2) and tight junction proteins such as claudin 1 (CLDN1) and tight junction protein 1 (TJP1) while suppressing inflammation (35). To examine the impact of the butyrate/BIX01294 combination on barrier function and inflammation, HTC cells were treated with butyrate and BIX01294 individually or in combination for 24 h, followed by a 3-h stimulation with LPS. To our surprise, although BIX01294 had a minimum activity in increasing CLDN1 expression, a marked synergy was observed in response to a combination of BIX01294 and butyrate, and the synergy was further enhanced in response to subsequent LPS treatment (FIG. 8, Panel A). However, MUC2 (FIG. 8, Panel B) and TJP1 (data not shown) were minimally affected in HTC cells by the compounds or their combination with or without LPS, while it is desirable that LPS had no impact on the AvBD9-inducing synergy (FIG. 8, Panel C).

Interestingly, although both butyrate and BIX01294 had no or weak activity in inducing gene expressions of inflammatory cytokines IL-1β (FIG. 8, Panel D) and IL-8 (FIG. 8, Panel E), the combination tended to have a synergistic effect on triggering the synthesis of both cytokines. While butyrate suppressed LPS-induced IL-1β expression, BIX01294 had an opposite effect, and a combination of butyrate and BIX01294 led to a significant reduction in IL-1B expression (FIG. 8, Panel D). Both butyrate and BIX01294 potentiated LPS-induced IL-8 expression individually, and the IL-8 expression level remained elevated in response to the combination (FIG. 8, Panel E). It is important to note that CLDN1 was also synergistically induced by butyrate and UNC1999 or by mocetinostat and UNC1999 (FIG. 8, Panel F), indicating that a combination of HDACi and HMTi is synergistic in augmenting CLDN1 gene expression; however, MUC2 and TJP1 genes were minimally regulated by HDACi and HMTi (data not shown).

### DISCUSSION

Enhancing the synthesis of endogenous HDPs has been shown to be effective against microbial infections and, therefore, is actively explored as a novel antibiotic-free, host-directed approach to antimicrobial therapy (9-11). Recently, a number of HDACi, HMTi, and DNMTi with the ability to induce HDP gene expression were identified from several high throughput screening efforts (21, 22) (and unpublished results). In this Example, a strong synergy was confirmed between three structurally distinct HDACi (butyrate, vorinostat, or mocetinostat) and three HMTi (BIX01294, A-366, or UNC1999) or two DNMTis (SGI-1027 or AZA, FIG. 1) in HDP gene induction in two chicken macrophage cell lines and PBMCs (Table 1 and the Figures). Additionally, most HDP genes appear to be upregulated by two-compound combinations, demonstrating the potential of these epigenetic compounds for disease control and prevention.

TABLE 1
Synergistic Induction of Chicken AvBD9 Gene by Combinations
of Histone Deacetylase Inhibitors (HDACi) and DNA/Histone
Methyltransferase Inhibitors (HMTi/DNMTi)

| | HDACi | | |
|---|---|---|---|
| | Butyrate | Vorinostat | Mocetinostat |
| HMTi | | | |
| BIX01294 | Yes | Yes | Yes |
| A-366 | Yes | Not Tested | Yes |
| UNC1999 | Yes | Not Tested | Yes |
| DNMTi | | | |
| SGI-1027 | Yes | Not Tested | Yes |
| 5-Azacytidine (AZA) | Yes | Not Tested | Yes |

“Yes” means that synergism was observed for the combination

Gene transcription is controlled by not only the DNA sequence, but also the accessibility of a gene promoter, while the latter is dictated by the modification status of histones and DNA in the region (12, 13). Among major epigenetic modifications of histones are acetylation and methylation (36, 37). The lysine residues in the amino-terminal tail of histones can be acetylated by HAT and deacetylated by HDAC. Acetylation of the lysine residues reduces the positive charge of the histones and subsequent binding of the histone tails with negatively charged DNA, thereby leading to a more relaxed and accessible chromatin, which facilitates the binding of transcription factors and transcriptional machinery for subsequent gene transcription (36, 37). Additionally, acetylated lysines also recruit bromodomain-containing transcription factors to the gene promoter for active transcription (38, 39). On the other hand, histone deacetylation leads to an opposite outcome with a more condensed chromatin and decreased gene transcription. Among four classes of HDAC, HDAC1 is primarily responsible for the constitutive expression of human β-defensin 1 (DEFB1) in the A549 lung epithelial cell line (40). HDACi function to maintain the acetylation status of chromatin and thereby facilitate gene transcription (41). A number of structurally different HDACi such as butyrate and entinostat have been found to have the HDP-inducing activity (10, 16).

Histones can also be methylated on the lysine or arginine residues by HMT or demethylated by histone demethylases (14). Histone methylation can lead to either an increase or decrease in gene transcription, depending largely upon the site of methylation. For example, methylation of lysine 4 of histone 3 (H3K4) leads to transcriptional activation, while methylation of H3K9, H3K20, and H3K27 signals transcriptional suppression (14). G9a, also known as euchromatic histone-lysine N-methyltransferase 2 (EHMT2), catalyzes methylation of H3K9 and H3K27 leading to gene silencing, while enhancer of zeste homolog 1/2 (EZH1/2) are another class of HMT that primarily catalyze methylation of H3K27 to suppress gene transcription (42). Trimethylation of H3K27 has been found to be negatively associated with HDP synthesis in human skin keratinocytes (20). Therefore, it is possible that inhibitors of G9a or EZH1/2 HMT could potentially lead to enhanced HDP synthesis.

Similarly, DNA can also be methylated at the C5 position of CpG dinucleotides by DNMT often leading to gene silencing by impairing the binding of transcription activators or facilitating the binding of transcription suppressors with high affinity for methylated CpG (14). Hypermethylation of the gene promoters has been found to be associated with reduced expression of two human β-defensin genes (DEFB1 and DEFB4) in prostate cancer cells (43) and oral carcinoma cells (17), respectively. Conversely, treatment of human oral carcinoma cells (17), vaginal keratinocyte cells (44), or bovine mammary epithelial cells (19) with a DNMTi induces HDP gene expression.

In this Example, three structurally distinct HDACi (butyrate, vorinostat, and mocetinostat), three HMTi (BIX01294, A-366, and UNC1999), and two DNMTi (SGI-1027 and 5-azacytidine) were used to evaluate their synergy (FIG. 1). Consistent with earlier reports, these epigenetic compounds are capable of inducing HDP gene expression in chicken HTC macrophages, demonstrating that these genes can be regulated by histone acetylation and DNA/histone methylation. Importantly, a dramatic synergy in induction of most HDP genes was observed in response to a combination of HDACi and HMTi or a combination of HDACi and DNMTi, implying therapeutic and prophylactic potential for these compounds against infectious diseases. Follow-up animal studies will confirm the in vivo efficacy of these compounds in disease control and prevention.

It is noted that butyrate, vorinostat (also known as suberoylanilide hydroxamic acid or SAHA), and mocetinostat are all pan-HDACi capable of inhibiting multiple classes of HDAC (45). It will be important to study relative HDP-inducing potency of those HDAC inhibitors that are specific for a certain class of HDAC. Given the major involvement of HDAC1 in human DEFB1 expression, HDACi with a strong ability to suppress HDAC1 may be potent in HDP gene induction (40). However, further investigations are warranted on whether such a mechanism is conserved among individual HDP genes across different animal species. While BIX01294 and A-366 are G9a-specific HMTi responsible mainly for suppression of H3K9 methylation (46), UNC1999 is an EZH1/EZH2-specific inhibitor that specifically suppresses H3K27 methylation (47). It is expected all HMTi that can alleviate repressive methylation marks of histones (e.g., H3K9 and H3K27) will induce HDP genes, while those HMTi that suppress transcriptionally active methylation marks (e.g., H3K4) will not.

The mechanism of the synergy in HDP gene induction between HDACi and HMTi or between HDACi and DNMTi remains to be studied. However, cross-talk between epigenetic modifications is known in controlling gene transcription. For example, methylated CpG-binding protein 2 (MeCP2) has a high affinity for methylated DNA, which in turn recruits HDAC leading to histone deacetylation, chromatin compaction, and gene silencing (48). Additionally, MeCP2 has been found to associate with HMT, causing H3K9 methylation leading to gene suppression (49). While not wishing to be bound by a particular theory, it is plausible that HDACi and DNMTi/HMTi could synergize chromatin relaxation and gene transcription. Surprisingly, combination therapies involving different classes of epigenetic modifiers are being explored for treatment of different types of cancers (50, 51). It is possible there is a link between induced HDP expression and enhanced apoptosis of tumor cells in response to combinations of epigenetic modifiers. Nevertheless, the discovery of the synergistic action in HDP gene induction between HDACi and DNMTi/HMTi leads to the development of a novel host-directed antimicrobial therapy against infectious diseases in accordance with the present disclosure.

### NON-LIMITING ILLUSTRATIVE EMBODIMENTS

The following is a list of non-limiting illustrative embodiments disclosed herein:

Illustrative embodiment 1. A composition, comprising: (a) at least one histone deacetylase inhibitor; and (b) at least one methyltransferase inhibitor selected from a DNA methyltransferase inhibitor and/or a histone methyltransferase inhibitor.

Illustrative embodiment 2. The composition of illustrative embodiment 1, wherein the at least one histone deacetylase inhibitor of (a) is selected from the group consisting of butyrate, phenylbutyrate, 4-phenylbutyric acid, β-hydroxybutyrate, vorinostat (suberoylanilide hydroxamic acid, SAHA), entinostat (MS-275), panobinostat (LBH589), mocetinostat (MGCD0103), belinostat (PXD101), romidepsin (FK228, depsipeptide), MC1568, tubastatin A, givinostat (ITF2357), dacinostat (LAQ824), CUDC-101, quisinostat (JNJ-26481585), pracinostat (SB939), PCI-34051, diallyl disulfide, depudecin, droxinostat, abexinostat (PCI-24781), RGFP966, AR-42, ricolinostat (ACY-1215), psammaplin A, tacedinaline (CI994), fimepinostat (CUDC-907), M344, tubacin, RG2833 (RGFP109), resminostat, BRD3308, SIS17, SR-4370, pimelic diphenylamide 106, NKL 22, azumamide E, tinostamustine (EDO-S101), SKLB-23bb, TH34, suberohydroxamic acid, UF010, WT161, valproic acid, 2-hexyl-4-pentynoic acid, tucidinostat (Chidamide), TMP195, TMP269, BRD73954, BG45, domatinostat (4SC-202), CAY10603, CAY10398, LMK-235, splitomicin, santacruzamate A (CAY10683), nexturastat A, HPOB, curcumin, divalproex sodium, scriptaid, GSK3117391, BML-210 (CAY10433), sulforaphane, raddeanin A, isoguanosine, sinapinic acid, tasquinimod, parthenolide, m-carboxycinnamic acid bis-hydroxamide, MC1742, JNJ-26481585, apicidin, HC toxin, oxamfaltin, bufexamac, KD5170, dihydrochlamydocin, 1-alaninechlamydocin, trapoxin A, microsporin A, largazole, sirtinol, inauhzin, SRT1720, SRT2104, SRT3025, CAY10591, CAY10602, thiomyristoyl, AGK2, JGB1741, salermide, AK-7, JFD00244, BML-278, pinosylvin, theobromine, EX-527, piceatannol, fisetin, vitexin, orientin, hyperforin, butein, ursolic acid, epicocconigrone, kaempferol, marein, zerumbone, lycorine, (S)-allylmercaptocysteine, and combinations thereof.

Illustrative embodiment 3. The composition of illustrative embodiment 1 or 2, wherein (b) comprises at least one DNA methyltransferase inhibitor selected from the group consisting of decitabine, azacitidine, 2′-deoxy-5-fluorocytidine, thioguanine, zebularine, gamma-oryzanol, β-thujaplicin, procainamide, bobcat339, SGI-1027, RG108, RSC-133, SW155246, genistein, ellagic acid, rosmarinic acid, and combinations thereof.

Illustrative embodiment 4. The composition of any of illustrative embodiments 1-3, wherein (b) comprises at least one histone methyltransferase inhibitor selected from the group consisting of BIX01294, UNC0224, UNC0321, UNC0631, UNC0638, UNC0642, UNC0646, UNC1999, UNC6852, BRD9539, BRD4770, sinefungin, tazemetostat, nanaomycin A, lirametostat, 3-deazaneplanocin A, chaetocin, A-366, EPZ005687, EPZ011989, EPZ6438, GSK126, GSK343, GSK503, CM-272, CPI-169, CPI-360, EBI-2511, MS1943, JQEZ05, El1, (R)-PFI-2, PF-06726304, ZLD1039, and combinations thereof.

Illustrative embodiment 5. The composition of any of illustrative embodiments 1-4, further comprising a pharmaceutically acceptable carrier or excipient.

Illustrative embodiment 6. The composition of illustrative embodiment 5, wherein the pharmaceutically acceptable carrier or excipient is aqueous.

Illustrative embodiment 7. The composition of illustrative embodiment 5, wherein the pharmaceutically acceptable carrier or excipient is oil-based.

Illustrative embodiment 8. The composition of any of illustrative embodiments 1-7, further defined as a nutritional composition.

Illustrative embodiment 9. The composition of illustrative embodiment 8, wherein the nutritional composition is an animal feed.

Illustrative embodiment 10. The composition of illustrative embodiment 8 or 9, wherein the nutritional composition is a nutritional supplement.

Illustrative embodiment 11. The composition of any of illustrative embodiments 1-10, further comprising at least one additional ingredient selected from the group consisting of a thickener, a flavoring, a diluent, an emulsifier, a dispersing aid, a bulking agent, a binder, and combinations thereof.

Illustrative embodiment 12. The composition of any of illustrative embodiments 1-11, wherein (a) is butyrate, and (b) is selected from the group consisting of BIX01294, A-366, and UNC1999.

Illustrative embodiment 13. The composition of any of illustrative embodiments 1-12, wherein (a) is butyrate, and (b) is selected from the group consisting of SGI-1027 and azacytidine.

Illustrative embodiment 14. The composition of any of illustrative embodiments 1-13, wherein (a) is vorinostat, and (b) is BIX01294.

Illustrative embodiment 15. The composition of any of illustrative embodiments 1-14, wherein (a) is mocetinostat, and (b) is of BIX01294, A-366, and UNC1999.

Illustrative embodiment 16. The composition of any of illustrative embodiments 1-15, wherein (a) is mocetinostat, and (b) is selected from the group consisting of SGI-1027 and azacytidine.

Illustrative embodiment 17. A method of inducing expression of at least one antimicrobial host defense peptide, the method comprising the step of contacting at least one cell with the composition of any one of illustrative embodiments 1-16 under conditions that allow for expression of the at least one antimicrobial host defense peptide.

Illustrative embodiment 18. The method of illustrative embodiment 17, wherein the at least one antimicrobial host defense peptide is selected from the group consisting of AvBD1, AvBD2, AvBD3, AvBD4, AvBD5, AvBD6, AvBD7, AvBD8, AvBD9, AvBD10, AvBD14, CATH2, CATHB1, and combinations thereof.

Illustrative embodiment 19. A method, comprising the step of administering to an animal the composition of any one of illustrative embodiments 1-16.

Illustrative embodiment 20. A method, comprising the step of: administering to an animal, either simultaneously or wholly or partially sequentially: (a) at least one histone deacetylase inhibitor; and (b) at least one methyltransferase inhibitor selected from a DNA methyltransferase inhibitor and/or a histone methyltransferase inhibitor.

Thus, in accordance with the present disclosure, there have been provided compositions, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
1. Schrader S M, et al. Sci Transl Med (2020) 12: eaaz6992.
2. Vidovic N, et al. Antibiotics (Basel) (2020) 9:52.
3. Magana M, et al. Lancet Infect Dis (2020) 20:e216-30.
4. Li W, et al. Chem Soc Rev (2021) 50:4932-73.
5. Cuperus T, et al. Dev Comp Immunol (2013) 41:352-69.
6. Zhang G, et al. Pharmaceuticals (Basel) (2014) 7:220-47.
7. Mansour S C, et al. Trends Immunol (2014) 35:443-50.
8. Ting D S J, et al. Front Immunol (2020) 11:983.
9. Bergman P, et al. Front Immunol (2020) 11:1209.
10. Robinson K, et al. Anim Nutr (2018) 4:160-9.
11. Wu J, et al. Adv Nutr (2020) 11:92-102.
12. Lawrence M, et al. Trends Genet (2016) 32:42-56.
13. Klemm S L, et al. Nat Rev Genet (2019) 20:207-20.
14. Michalak E M, et al. Nat Rev Mol Cell Biol (2019) 20:573-89.
15. Ho T C S, et al. J Med Chem (2020) 63:12460-84.
16. Rodriguez-Carlos A, et al. Peptides (2021) 140:170527.
17. Kamino Y, et al. Oncol Rep (2014) 32:462-8.
18. Chen X, et al. Oncotarget (2017) 8:27943-52.
19. Kweh M F, et al. J Dairy Sci (2019) 102:5706-12.
20. Gschwandtner M, et al. J Invest Dermatol (2014) 134:2192-201.
21. Deng Z, et al. J Immunol Res (2018) 2018:5492941.
22. Lyu W, et al. Front Cell Infect Microbiol (2018) 8:191.
23. Wang J, et al. J Anim Sci Biotechnol (2021) 12:14.
24. Rath N C, et al. Vet Immunol Immunopathol (2003) 96:93-104.
25. Beug H, et al. Cell (1979) 18:375-90.
26. Sunkara L T, et al. PloS One (2011) 6:e27225.
27. Robinson K, et al. Front Nutr (2021) 8:778424.
28. Yang Q, et al. Front Microbiol (2021) 12:781649.
29. Yang Q, et al. Antibiotics (Basel) (2021) 10:1175.
30. Yang Q, et al. J Anim Sci Biotechnol (2022) 13:3.
31. Schmittgen T D, et al. Nat Protoc (2008) 3:1101-8.
32. Schauber J, et al. Gut (2003) 52:735-41.
33. Yang Q, et al. Front Immunol (2022) 13:819222.
34. Zhou Z, et al. Curr Top Med Chem (2018) 18:2448-57.
35. Salvi P S, et al. Cells (2021) 10:1775.
36. Zhang Q, et al. Nat Rev Immunol (2019) 19:417-32.
37. Zhao Z, et al. Genome Biol (2019) 20:245.
38. Ramadoss M, et al. Drug Discov Today (2018) 23:76-89.
39. Jostes S, et al. Nat Rev Urol (2019) 16:245-59.
40. Kallsen K, et al. PloS One (2012) 7:e50000.
41. Barnes C E, et al. Essays Biochem (2019) 63:97-107.
42. Richart L, et al. Curr Opin Chem Biol (2020) 56:51-62.
43. Lee J, et al. PloS One (2016) 11:e0166664.
44. Lee J, et al. Probiotics Antimicrob Proteins (2017) 9:406-14.
45. Eckschlager T, et al. Int J Mol Sci (2017) 18:1414.
46. Ciechomska I A, et al. Sci Rep (2016) 6:38723.
47. Xu B, et al. Blood (2015) 125:346-57.
48. Nan X, et al. Nature (1998) 393:386-9.
49. Fuks F, et al. J Biol Chem (2003) 278:4035-40.
50. Yen C Y, et al. Cancer Lett (2016) 373:185-92.
51. Zahnow C A, et al. Adv Cancer Res (2016) 130:55-111.
52. Bhat A A, et al. Int J Mol Sci (2020) 21:569.
53. Liu Y, et al. Int J Biol Macromol (2020) 164:884-91.

1. A composition, comprising:
(a) at least one histone deacetylase inhibitor; and
(b) at least one methyltransferase inhibitor selected from a DNA methyltransferase inhibitor and/or a histone methyltransferase inhibitor.

2. The composition of claim 1, wherein the at least one histone deacetylase inhibitor of (a) is selected from the group consisting of butyrate, phenylbutyrate, 4-phenylbutyric acid, β-hydroxybutyrate, vorinostat (suberoylanilide hydroxamic acid, SAHA), entinostat (MS-275), panobinostat (LBH589), mocetinostat (MGCD0103), belinostat (PXD101), romidepsin (FK228, depsipeptide), MC1568, tubastatin A, givinostat (ITF2357), dacinostat (LAQ824), CUDC-101, quisinostat (JNJ-26481585), pracinostat (SB939), PCI-34051, diallyl disulfide, depudecin, droxinostat, abexinostat (PCI-24781), RGFP966, AR-42, ricolinostat (ACY-1215), psammaplin A, tacedinaline (CI994), fimepinostat (CUDC-907), M344, tubacin, RG2833 (RGFP109), resminostat, BRD3308, SIS17, SR-4370, pimelic diphenylamide 106, NKL 22, azumamide E, tinostamustine (EDO-S101), SKLB-23bb, TH34, suberohydroxamic acid, UF010, WT161, valproic acid, 2-hexyl-4-pentynoic acid, tucidinostat (Chidamide), TMP195, TMP269, BRD73954, BG45, domatinostat (4SC-202), CAY10603, CAY10398, LMK-235, splitomicin, santacruzamate A (CAY10683), nexturastat A, HPOB, curcumin, divalproex sodium, scriptaid, GSK3117391, BML-210 (CAY10433), sulforaphane, raddeanin A, isoguanosine, sinapinic acid, tasquinimod, parthenolide, m-carboxycinnamic acid bis-hydroxamide, MC1742, JNJ-26481585, apicidin, HC toxin, oxamfaltin, bufexamac, KD5170, dihydrochlamydocin, 1-alaninechlamydocin, trapoxin A, microsporin A, largazole, sirtinol, inauhzin, SRT1720, SRT2104, SRT3025, CAY10591, CAY10602, thiomyristoyl, AGK2, JGB1741, salermide, AK-7, JFD00244, BML-278, pinosylvin, theobromine, EX-527, piceatannol, fisetin, vitexin, orientin, hyperforin, butein, ursolic acid, epicocconigrone, kaempferol, marein, zerumbone, lycorine, (S)-allylmercaptocysteine, and combinations thereof.

3. The composition of claim 1, wherein (b) comprises at least one DNA methyltransferase inhibitor selected from the group consisting of decitabine, azacitidine, 2′-deoxy-5-fluorocytidine, thioguanine, zebularine, gamma-oryzanol, β-thujaplicin, procainamide, bobcat339, SGI-1027, RG108, RSC-133, SW155246, genistein, ellagic acid, rosmarinic acid, and combinations thereof.

4. The composition of claim 1, wherein (b) comprises at least one histone methyltransferase inhibitor selected from the group consisting of BIX01294, UNC0224, UNC0321, UNC0631, UNC0638, UNC0642, UNC0646, UNC1999, UNC6852, BRD9539, BRD4770, sinefungin, tazemetostat, nanaomycin A, lirametostat, 3-deazaneplanocin A, chaetocin, A-366, EPZ005687, EPZ011989, EPZ6438, GSK126, GSK343, GSK503, CM-272, CPI-169, CPI-360, EBI-2511, MS1943, JQEZ05, El1, (R)-PFI-2, PF-06726304, ZLD1039, and combinations thereof.

5. The composition of claim 1, further comprising a pharmaceutically acceptable carrier or excipient.

6. The composition of claim 5, wherein the pharmaceutically acceptable carrier or excipient is aqueous.

7. The composition of claim 5, wherein the pharmaceutically acceptable carrier or excipient is oil-based.

8. The composition of claim 1, further defined as a nutritional composition.

9. The composition of claim 8, wherein the nutritional composition is an animal feed.

10. The composition of claim 8, wherein the nutritional composition is a nutritional supplement.

11. The composition of claim 8, further comprising at least one additional ingredient selected from the group consisting of a thickener, a flavoring, a diluent, an emulsifier, a dispersing aid, a bulking agent, a binder, and combinations thereof.

12. The composition of claim 1, wherein (a) is butyrate, and (b) is selected from the group consisting of BIX01294, A-366, and UNC1999.

13. The composition of claim 1, wherein (a) is butyrate, and (b) is selected from the group consisting of SGI-1027 and azacytidine.

14. The composition of claim 1, wherein (a) is vorinostat, and (b) is BIX01294.

15. The composition of claim 1, wherein (a) is mocetinostat, and (b) is of BIX01294, A-366, and UNC1999.

16. The composition of claim 1, wherein (a) is mocetinostat, and (b) is selected from the group consisting of SGI-1027 and azacytidine.

17. A method of inducing expression of at least one antimicrobial host defense peptide, the method comprising the step of:
contacting at least one cell with the composition of claim 1 under conditions that allow for expression of the at least one antimicrobial host defense peptide.

18. The method of claim 17, wherein the at least one antimicrobial host defense peptide is selected from the group consisting of AvBD1, AvBD2, AvBD3, AvBD4, AvBD5, AvBD6, AvBD7, AvBD8, AvBD9, AvBD10, AvBD14, CATH2, CATHB1, and combinations thereof.

19. A method, comprising the step of:
administering to an animal the composition of claim 1.

20. A method, comprising the step of:
administering to an animal, either simultaneously or wholly or partially sequentially:
(a) at least one histone deacetylase inhibitor; and
(b) at least one methyltransferase inhibitor selected from a DNA methyltransferase inhibitor and/or a histone methyltransferase inhibitor.
